(19) United States
(12) Patent Application Publication
(10) Pub. No.: US 2018/0369289 A1
(43) Pub. Date: Dec. 27, 2018

(54) CELL TRANSPLANTATION DEVICE

(71) Applicant: THE REGENTS OF THE UNIVERSITY OF CALIFORNIA, Oakland, CA (US)

(72) Inventors: Jonathan R.T. Lakey, Irvine, CA (US); Michael Alexander, Oakland, CA (US); Rahul Krishnan, SANTA ANA, CA (US)

(73) Assignee: THE REGENTS OF THE UNIVERSITY OF CALIFORNIA, Oakland, CA (US)

(21) Appl. No.: 15/747,081

(22) PCT Filed: Aug. 3, 2016
(86) PCT No.: PCT/US2016/045427

§ 371 Date: Jan. 23, 2018

Related U.S. Application Data
(60) Provisional application No. 62/200,938, filed on Aug. 4, 2015.

(51) Int. Cl. A61K 35/39; A61K 31/555; A61K 35/28; A61K 35/35; A61K 33/40; A61K 35/407; A61K 38/18; A61K 9/50; A61K 38/36; A61K 38/44; A61K 38/48; A61P 3/10

(52) U.S. Cl. CPC A61K 35/39; A61K 31/555; A61K 35/28; A61K 35/35; A61K 33/40; A61K 35/407; A61K 2300/00; A61K 9/5036; A61K 38/1866; A61K 38/363; A61K 38/44; A61K 38/4886; A61P 3/10; A61K 38/1825

(57) ABSTRACT

The invention provides devices and associated methods for transplanting cells within the body. In some embodiments, the invention relates to the transplantation of insulin-producing cells including, for example, islet cells. In other embodiments, the cells are encapsulated prior to implantation. The encapsulation system and device optionally may contain one or more biologically active substances including, for example, an immunorepellant, an angiogenic protein, and/or a particulate oxygen generating substance.

### CROSS-REFERENCE TO RELATED APPLICATIONS

This application claims benefit of U.S. provisional application 62/200,938, filed August 4, 2015, which is hereby incorporated by reference in its entirety.

### FIELD OF THE INVENTION

The invention relates to devices and associated methods for transplanting cells within the body and insulin-producing cells that may be implanted by any means, including in combination with the disclosed devices.

### BACKGROUND

Diabetes is the fourth leading cause of death in the United States with more than 3 million Americans currently suffering from Type 1 diabetes (“T1D”). A promising treatment for T1D is the transplantation of donor islets to restore euglycemia (e.g. a normal level of sugar in the blood). However, clinical islet transplantation currently requires a lifetime of immune-suppression therapy and is encouraged only for diabetic patients with life-threatening complications. Therefore, encapsulation of islet tissue is a promising strategy that prevents direct contact between implanted islet cells and the host's immune system that may allow patients to receive islet transplants without requiring lifelong immunosuppressive therapy.

Encapsulation of islets within permeable or selectively permeable hydrogels provides a partial immune barrier and precludes the need for pharmacological immunosuppression for allografts. Advances in encapsulation technology have augmented islet viability (>80%) and graft function while reducing the minimal required curative islet dose.

Native islets demonstrate an extensive vascular network of capillaries a condition not satisfied in grafts which limits oxygen supply and attenuates graft function, especially in encapsulated islets. Encapsulated islets depend primarily on oxygen diffusion across relatively large distances as their vascular access is restricted and limited by structural dimensions of the device. Measured oxygen concentrations at the transplant site (around 60 mm Hg at subcutaneous sites and 40 mm Hg at intraperitoneal sites) are also markedly lower than the arterial oxygen partial pressure (102 mm Hg). These factors may expose the islet to hypoxic or even anoxic conditions, and it has been shown that improving oxygen delivery to encapsulated islets has enhanced islet function and viability.

### SUMMARY OF THE INVENTION

The present invention provides devices, compositions, systems, and methods for encapsulating and transplanting insulin-producing cells, such as islet cells, for the treatment of diabetes. Relative to the prior art encapsulation technologies, the inventions disclosed herein provide (i) reduced host immune recognition and subsequent immune-mediated attack and destruction of the device and/or encapsulated cells, (ii) improved vascularization (blood supply) to the encapsulated cells, and/or (iii) improved oxygenation of the encapsulated cells. The encapsulation devices and compositions, and transplantation methods may be applied to other cell types that may be useful in therapeutic transplantation methods.

In one aspect, the invention provides a microcapsule suitable for implanting cells into a subject, a microcapsule loaded with implantable and therapeutic cells, and methods for making the same. The inventive microcapsules are made primarily or solely from an alginate. The alginate may be synthetically manufactured or purified/extract from natural sources (e.g., seaweeds from Laninara, Ascophyllum, Ecklenia, Lessonia, Macrocystis, Durvillaea, Phaeophyceae, Rhodophceae, and Chlorophyceae species.) The alginates may be provided as a salt with any appropriate cation (e.g., Na, K, Mg, and Ca) or modified with organic substituents such as propylene glycol (PG) and/or polyethylene glycol (PEG). The alginate may comprise one or more very low viscosity alginates, low viscosity alginates, medium viscosity alginates, or mixtures thereof. It is understood that alginate viscosity depends upon the identity of the alginate polymer, including the guluronate:mannuronate ratio and the polymer form (e.g., block size in a block co-polymer), concentration of the alginate solution and length of the alginate polymer chains (i.e., the average molecular weight), with longer chains (higher average molecular weight) resulting in higher viscosities. In some embodiments, the alginate capsules are formed from low viscosity alginates including, for example an alginate having at least 50%, 55%, 60%, 65%, 70%, 75%, or 80% guluronic acid. In other embodiments, the low viscosity alginate comprises more than 70%, 75%, 80%, 85%, 80, or 95% mannuronic acid.

In some embodiments, the alginate microcapsules have a mean diameter of about 250, 300, 350, 400, 450, 500, 550, 600 μm, or more including, for example, about 250-600 μm, 300-500 μm, 350-450 μm, and 200-400 μm.

The alginate microcapsules may be manufactured by (a) providing an aqueous solution comprising 1-7.5% alginate (e.g., about 1.5-5.0% and about 1.5-3.5%, including about 1.5%, 2.5%, and 3.5%) and (b) extruding microcapsules of the desired size into a cationic solution. The extrusion process may use an electrostatic generator. Optionally, the extrusion pressure is about 1.5 psi (e.g., about 2-4 psi). Extrusion may be performed using a nozzle head (e.g., a needle) of any appropriate size that results in the desired microcapsule size based on the composition of the aqueous solution. Suitable extrusion nozzles are about 20-30 G (e.g., about 22-28 G, 24-26 G, and including 22, 24, 25, 26, and 28 G). In some embodiments, the cationic solution contains a diavalent cation such as Ca, Mg, or Ba, optionally present as its chloride salt. The cation may be present at about 50-200 mM or about 100-150 mM, including about 50, 100, 120, 150, 170, and 200 mM. Optionally, the microcapsules are extruded into an air gap and allowed to fall into the cationic solution. The air gap (i.e., distance from the extruder to the cationic solution surface) may be about 10-40 mm including about 10, 15, 20, 25, 30, 35, and 40 mm.

In some embodiments, the alginate microcapsules further comprise one or more immunorepellant, one or more angiogenic molecule, one or more particulate oxygen generating substance (POGS), and/or one or more perfluorocarbon (PFC). These molecules may be incorporated into the microcapsule by dissolving the molecules in the aqueous alginate solution. The molecules may be present in any biologically effective amount. For example, an immunorepellant such as CXCL12 may be incorporated into the aqueous alginate solution at 0.5-5 μg/ml and/or an angiogenic molecule such as FGF-1 may be incorporated into the aqueous alginate solution at 0.5-5 μg/ml.

In some embodiments, the alginate capsule contains (a) only one or more immunorepellants, (b) only one or more angiogenic molecules, (c) only one or more POGS, (d) only one or more PFC, (e) at least one immunorepellant and at least one angiogenic molecule alone or in combination with a POGS and/or a PFC, (f) at least one immunorepellant and at least one POGS alone or in combination with a PFC; (g) at least one immunorepellant and at least one PFC; (h) at least one angiogenic molecule and at least one POGS alone or in further combination with a PFC; and (i) at least one POGS and at least one PFC.

In other embodiments, the alginate microcapsule further comprises a population of therapeutic cells. The therapeutic cells optionally may produce and/or secrete a biologically active molecule (e.g., insulin). The population may be pure (i.e. substantially a single biologically-active cell type) or a mixed (i.e., two, three, four, or more biologically-active cell types). Therapeutic cells include, for example, islet cells, hepatocytes, chondrocytes, and lipocytes (e.g., obtained or derived from human or porcine) and undifferentiated, partially differentiated, or fully differentiated stem cells (e.g., iPS, adult or embryonic stem cells). Stem cells may be derived from any suitable species and may (or may not) be genetically modified (e.g., comprising one or more transgenes). Suitable species include, for example, human, rat, mouse, and pig. Therapeutic cells may be introduced into the microcapsules by mixing the cells with the aqueous alginate solution prior to extrusion.

The invention also provides methods for treating diabetes in any species including, for example, humans, cats, dogs, horses, and cattle (esp. cows). First, the subject is diagnosed as having diabetes (e.g., Type 1 diabetes). Alginate microcapsules containing insulin-secreting cells (e.g., human or porcine islets, islet cells, or differentiated stem cells) are prepared as described herein. The alginate microcapsules are then surgically implanted into the subject. Implantation may be subcutaneous, intramuscular, intraperitoneal, or within the kidney capsule. In one embodiments, the blood glucose level (e.g., fasting blood glucose) of the subject is periodically measured. Blood glucose may be measured at least daily, weekly, monthly, or more infrequently. When the measured blood glucose level exceeds a pre-determined threshold (e.g., >350 mgl/dl for fast blood glucose), a new batch of alginate microcapsules is surgically implanted into the same or a different site. Optionally, the process is repeated for the life of the patient. In some embodiments, a dose of 2,500-10,000 IEQ/kg is implanted into the subject. The specific dose may be about 3,000-8,000 IEQ/kg, about 4,000-6,000 IEQ/kg. or at least about 2,500, 3,000, 4,000, 5,000, 6,000, 7,000, 8,000, 9,000, 10,000, IEQ/kg or more.

By “alginate” is meant an anionic polysaccharide that consists of β-D-mannuronate and α-L-guluronate copolymers (e.g., block copolymers). In some embodiments, the monomeric units are (1-4)-linked. In other embodiments, the alginate is a block co-polymer of β-D-mannuronate and α-L-guluronate. Alginate polymers may be linear or branched (e.g., PEG-branched alginates). In some embodiments, the alginate comprises more than 50%, 55%, 60%, 65%, 70%, 75%, or 80% guluronic acid. In other embodiments, the alginate comprises more than 70%, 75%, 80%, 85%, 80%, or 95% mannuronic acid.

By “very low viscosity alginates” is meant an alginate having a viscosity of <20 mPa·s at room temperature.

By “low viscosity alginates” is meant an alginate having a viscosity of 20-200 mPa·s at room temperature.

By “medium viscosity alginates” is meant an alginate having a viscosity of >200 mPa·s at room temperature.

By “immunorepellant” is meant any molecule (e.g., a protein) that locally suppresses at least one immunological function (e.g., repels leukocytes) at or near the site of administration. Immunorepellants include, for example, CXCL12, SDF-1, IL-8, and TGF-β.

By “angiogenic molecule” is meant any molecule (e.g., protein) that induces neo-vascularization at or near the site of administration. Angiogenic molecules include, for example, fibrin, FGF-1, FGF-2, VEGF, PDGF, and MMP.

By “particulate oxygen generating substance” (“POGS”) is meant any substance suitable for incorporation into an implantable device described herein that is capable of releasing molecular oxygen over time upon contact with water. POGS include, for example, inorganic peroxides (e.g., calcium peroxide) and inorganic percarbonates (e.g., sodium percarbonate).

By “PFC” or “perfluorocarbon” is meant any organoflourine that has a binding affinity for molecular oxygen. PFCS include, for example, perfluorodecalin, Oxygent® (Alliance Pharmaceuticals, Corp., San Diego, Calif.; 58% w/v perfluorooctyl bromide and 2% w/v perfluorodecyl bromide in a phospholipid emulsion), Oxyfluor® (Alliance Pharmaceuticals, Corp., San Diego, Calif.; perfluorodichlorooctane, egg yolk phospholipid and triglyceride), perfluorooctyl bromide, perfluoron, perfluorodecyl bromide, perfluorodichlorooctane, hemagen perfluorocarbon emulsion, perfluoro-octane, perfluoroperhydrophenanthrene, perfluorotributylamide, perfluorooctylbromide, hydrogenated hydrofluorocarbon liquids (e.g., F6 H6 , F6H8, O44, and O62), perfluorohexane, and Oxy-PDT nanoparticles (perfluorohexane and IR780 photosensitizer).

### DESCRIPTION OF DRAWINGS

FIG. 1 is a line graph showing the effect of time and incubation temperature on the size of 2.5% UPLVM alginate microcapsules, as described in Example 2. Alginate microcapsules were incubated at 3, 23, 37, or 43° C. for 7 days and then transferred to 3, 23, 37, or 43° C. for an additional 7 days. Capsule diameter was measured at 12 hours, 1, 3, 7, 11, and 14 days post capsule production.

FIG. 2 is a bar graph showing the effect of time and incubation temperature on the volume of alginate microcapsules, as described in Example 2. Alginate microcapsules were made at 23° C. then incubated for 14 days at 3, 23, 37, and 43° C. Capsule diameter was measured at 4 and 12 hours, 1, 7, and 14 days.

FIG. 3 is a line graph showing the effect time, incubation temperature, and alginate microcapsule composition on the volume of alginate microcapsules, as described in Example 2. Alginate microcapsules were made using 2.5% UPLVM or 2.5% UPLVG and were incubated for 7 days at 3 or 37° C. After 7 days, the capsules were either kept at their original temperature or switched to 3 or 37° C. Capsule diameter were measured at 0, 6, 12, 24, 72, 120, 168, 216, 264, and 360 hours.

FIG. 4 is a series of line graphs showing the effect of varying incubation temperatures on microcapsule permeability to dextrans of varying molecular weights. Alginate microcapsules were made using 2.5% UPLVM or 2.5% UPLVG and were incubated for 14 days at 3 or 37° C. Dextran (10, 70, 150, 250, and 500 kDa) diffusion was used to measure the capsule permeability.

FIG. 5 is a line graph illustrating the amount of dissolved oxygen in culture media after the addition of a POGS in either free- or encapsulated form.

FIG. 6 is a line graph illustrating the amount of dissolved oxygen in culture media after the addition of various POGS formulations.

FIG. 7 is a bar graph showing the viability of islets cultured under normoxic and hypoxic conditions in the presence and absence of a POGS.

FIG. 8 is a bar graph showing the GSIR from islets cultured under normoxic and hypoxic conditions in the presence and absence of a POGS.

FIG. 9A is a of line graph showing the time course of PFC oxygenation in culture media under normoxic conditions. FIG. 9B is a line graph showing the PFC oxygen release profile in culture media under hypoxic conditions.

FIG. 10 is a bar graph showing the survival of cultured islets under normoxic and hypoxic conditions in the presence of various PFC concentrations.

FIG. 11 a pair of photomicrographs showing macrophage attachment to a 2.5% UPLVM microcapsule lacking CXCL12/SLF-1 (right) but no macrophage attachment to a similar capsule containing CXCL12/SLF-1 (left).

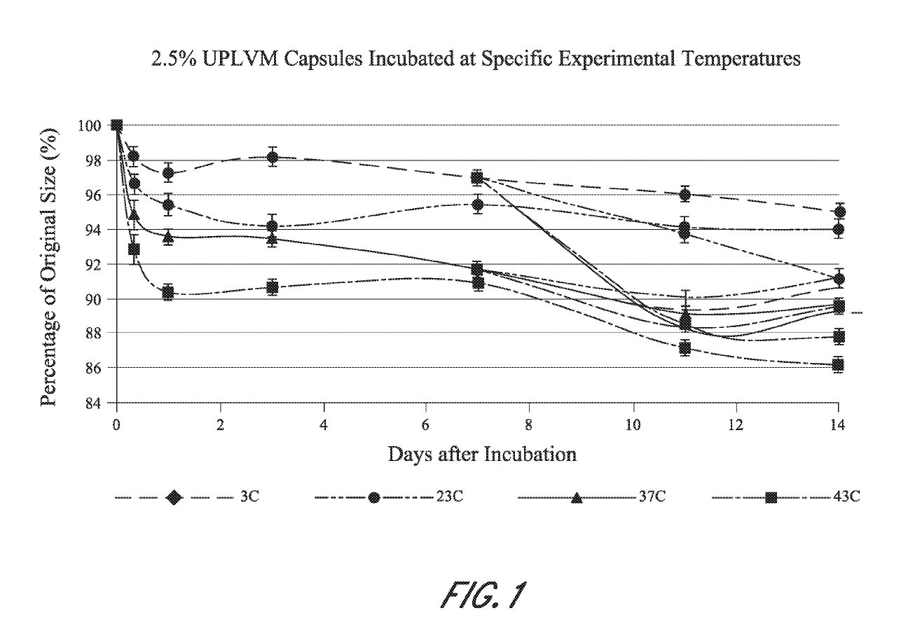

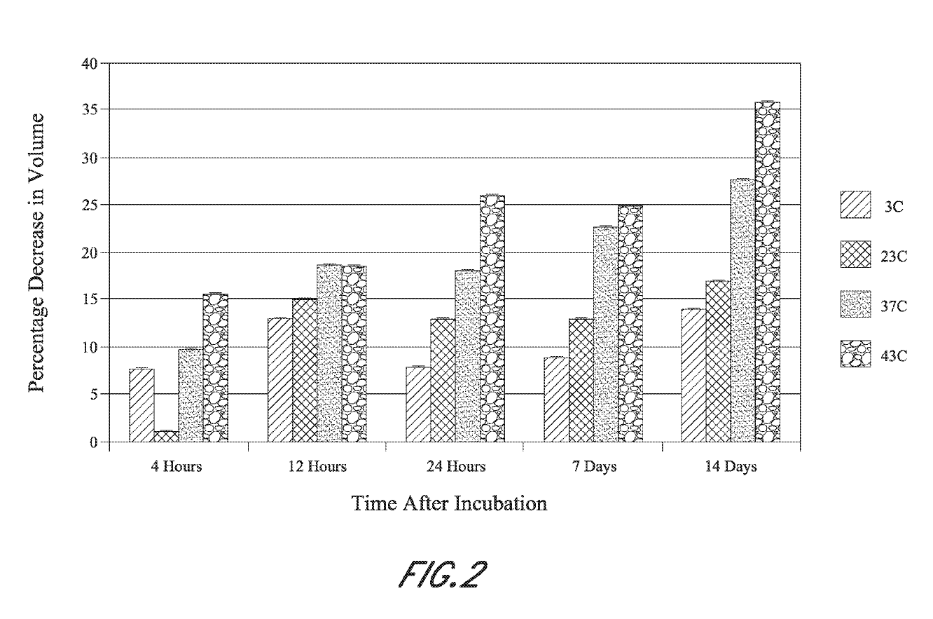

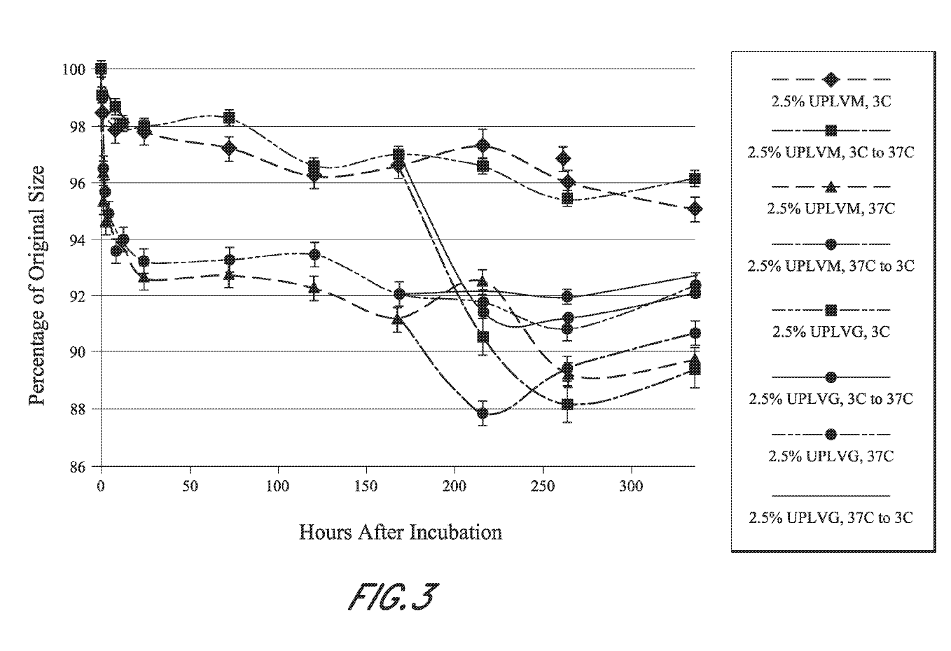

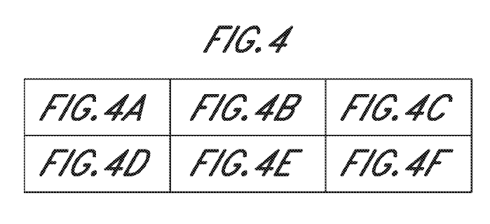

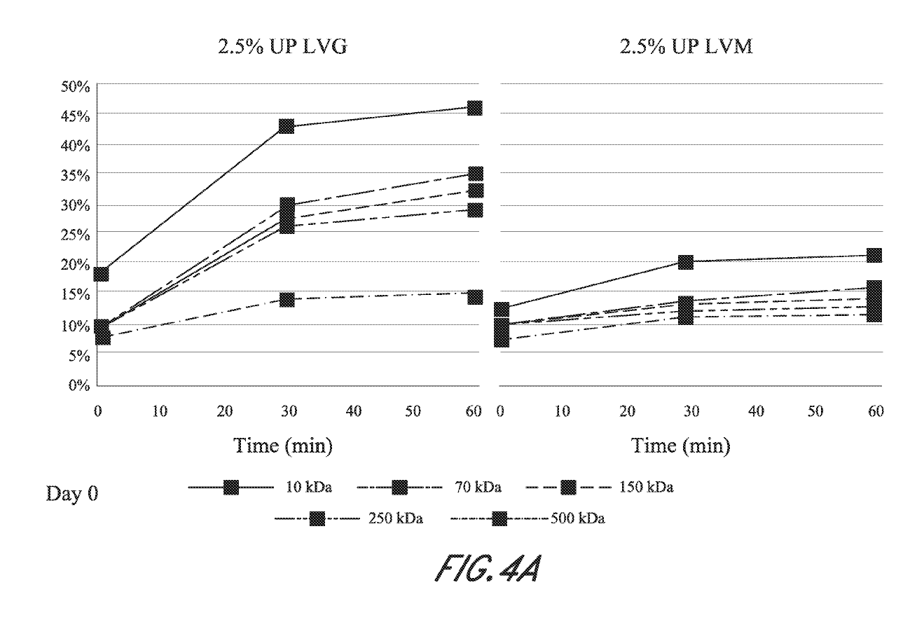

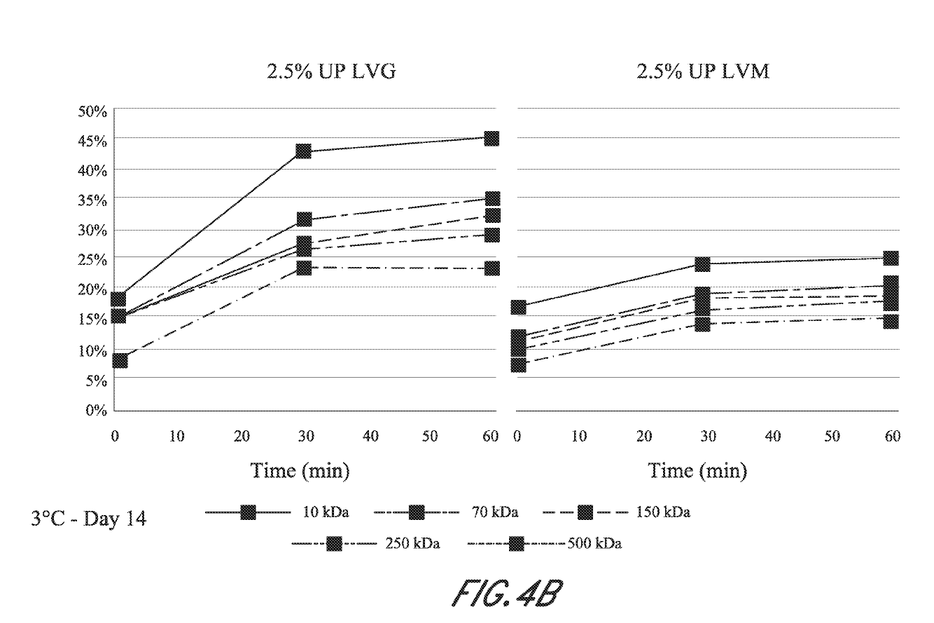

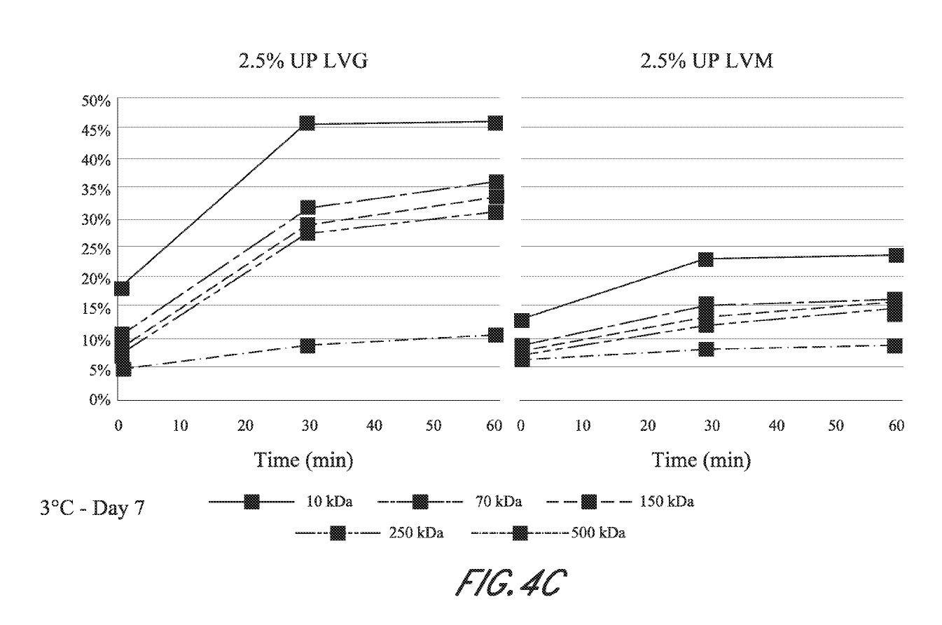

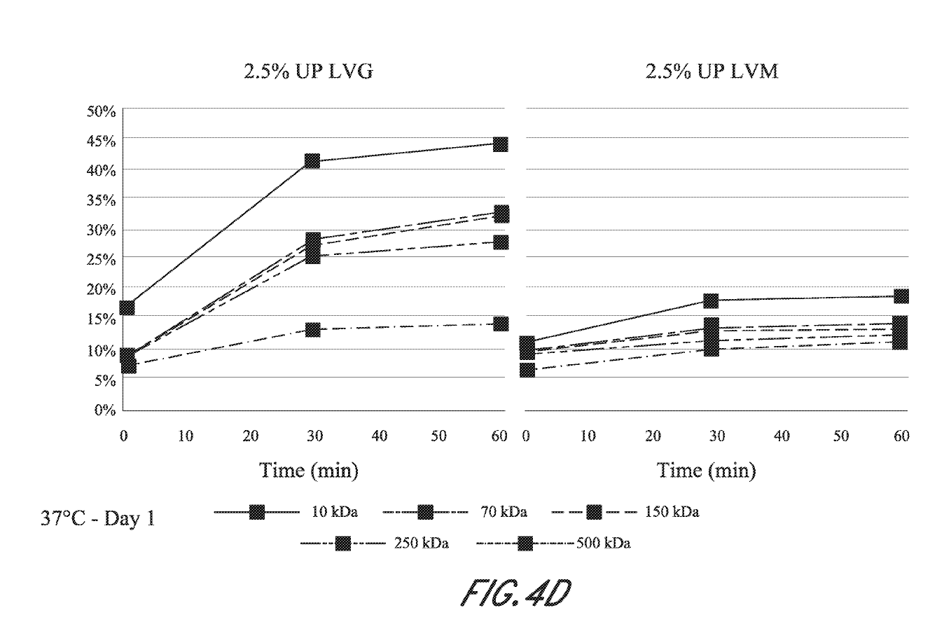

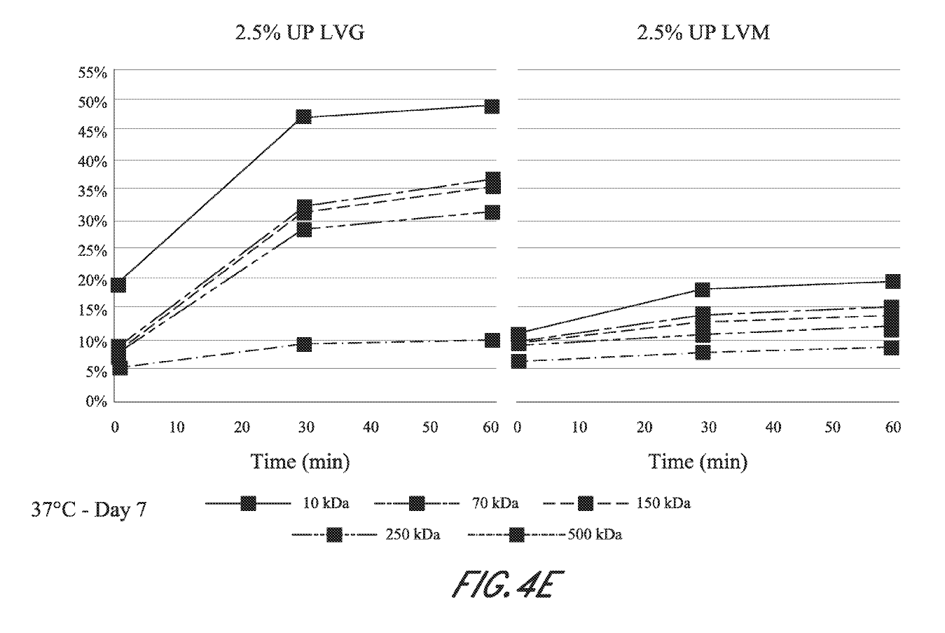

### DETAILED DESCRIPTION

Over the last decade, human islet transplantation has emerged as a viable alternative to conventional management strategies to treat type 1 diabetes. With appropriate immunosuppression regimens, patients that are able to remain insulin independent for up to 8 years post-transplant. Encapsulation of islets within biocompatible hydrogels is a solution proposed by biomedical engineers to address this uniquely frustrating issue. Cell encapsulation involves enveloping cells in a selective permeable biocompatible matrix that allows for the diffusion of oxygen and nutrients but is able to effectively prevent immune cells and antibodies from reaching the graft, thus delaying rejection. Encapsulation can be employed as a platform to deliver localized immunosuppression at the transplant site, thus avoiding the adverse affects of chronic systemic immunosuppression and can also be used to deliver nutrients and biological agents that will enhance islet survival and functions after transplantation into patients. While it is clear that encapsulation has several advantages over conventional islet transplantation, several roadblocks currently prevent translation of results from small animal and primate trials to human trials. Alginate pore size is a crucial parameter that aids in the protection of islets from host immune recognition, while allowing insulin, oxygen, and other micronutrients to diffuse through the capsules. Islet encapsulation within alginate hydrogels is advantageous because it prevents direct contact between the encapsulated islets and the host immune system while significantly reducing the need for chronic systemic immunosuppression.

The invention provides islets and islets cells that may be transported for the treatment of diabetes, either as bare cells or encapsulated using any encapsulation platform, including the platforms disclosed herein, and method for making the same. The invention also provides alginate microcapsules and related production methods that may be used for transplanting any therapeutic cell type, including islets and islet cells. The invention also provides other implantable scaffold devices and related production methods that may be used for transplanting any therapeutic cell type, including islets and isle cells.

### EXAMPLE 1

The aim of the study was to characterize Juvenile porcine islets and evaluate insulin production, thereby B cell content, over a two week culture period. This knowledge provides insight on optimal islet use in xenotransplantation therapy studies for Type 1 Diabetes.

Islets were isolated and matured from Juvenile Yorkshire pigs using previously described techniques. (Lamb et al., Cell Transplantation, 23:263-272, 2014). Briefly, pancreases from Juvenile pigs (18-20 days) were rapidly procured, under 5 minutes, and placed in Eurocollins Solution (Cellgro, Manassas, Va.). Cold ischemia time was limited to less than 30 minutes. Tissue was then minced, followed by partial enzymatic digestion. 15 minutes at 37° C. shaking (70 rpm) water bath in low dose CIzyme collagenase MA/BP protease enzyme (VitaCyte, San Diego, Calif.). Islet tissue clusters were then cultured at 37° C./5% CO₂ in Maturation Media (Cellgro Manassas, Va.), with media changes every 48 hours.

Islet yield and purity was determined post-isolation through previously described methods. (Lamb et a., Cell Transplantation, 23:263-272, 2014). Samples of islets (200 μL) were incubated for 5 minutes with dithizone and then viewed under a microscope. Islet function was determined through previously described methods. (Green et al., Biochem. J., 134: 481-487, 1973: Ricordi et al., Acta Diabetol. Lat. 27: 185-195, 1990). Seventy five IEQ of islets were used per sample. Insulin was measured using ELISA (Marcodia, Sweden).

Samples were characterized for islet cellular composition on days, 3, 7 and 14 of culture. To characterize cellular composition during tissue culture, groups of 1000-1500 islets were washed and centrifuged (200°G) twice in Dulbecco phosphate buffered saline (DPBS, Cellgro, Manassas, Va.) followed by an 11-minute dissociation at 37° C. using Acurase (500-720 U/ml, Innovative Cell Technologies, San Diego, Calif.). Single cell suspensions were then filtered through a 40 μm filter to remove any debris. Suspensions were then transferred to flow cytometry tubes and stained with Fixable viability dye (E Fluor 506, eBioscience, San Diego, Calif.) for 30 minutes. Cells were then washed with DPBS and centrifuged at 1200 RPM for 5 minutes to remove the dye and fixed in 4% PFA for 5 minutes. Cells were washed with DPBS and centrifuged again, and then suspended in Protein Block (Abcam, Cambridge, UK) for 30 minutes. Cells were then washed and centrifuged followed by addition of specific fluorescently conjugated antibodies for cellular markers for 30 minutes. Amylase conjugated with FITC was used as a marker for acinar cells (Abcam, Cambridge, UK), and PE conjugated Insulin was used as a marker for β-cells (Cell Signaling Technology, Danvers, Mass.). Cell populations were then quantified using flow cytometry (MacsQuant, Miltenyi Biotec, San Diego, Calif.) by running samples at medium speed. An unstained control as well as single-stain controls was used to determine true stained populations. Cell populations were then analyzed for β and acinar cell content using FlowJo Software (Flowjo LLC, Ashland, Oreg.).

Viability: Samples of islets were tested on 3, 7 and 14 days post-isolation. Islet viability remained high throughout the 14-day tissue culture duration (83.8±2.0%, 78.1±3.4%, 81.9±1.6%; days, 3, 7, and 14, respectively; p=0.1, NS).

Islet Yield: The proportion of dithizone positive tissue significantly increased during tissue culture with 12,600±183 IEQ (mean±SEM) on day 0 up to 33,300±136 IEQ on day 7 (p<0.05), demonstrating that islet quantity significantly increased throughout the maturation process.

Glucose-stimulated Insulin Release Assay: Islet function significantly improved during in vitro culture until day 7 post digestion (SI=1.9±0.2; day 3, SI=2.6±0.5; day 7, p=0.02) after which no further change was noted (SI=2.3±1.2; Day 14, p=0.3, NS).

Flow Cytometry: During the first 7 days of islet maturation, acinar proportion decreased marginally (14.9±2.5%; Day 3, 16.4±2.1; Day 7, p=0.07), while β-cell proportion in islets increased significantly (7.7±1.8%; Day 3, 38.6±3.5%; Day 7, p=0.04). Following the first 7 days, changes in composition of the islets is nonsignificant. Acinar tissue concentration remained relatively constant (10.3±3.0%; Day 14 p>0.05) while β cell content decreased some (22.4±3.7%; Day 14, p>0.05).

This example provides a simple method for characterizing islets to monitor the progression of β cell maturation and purity of juvenile porcine islets. During the maturation process, an overall reduction in the percentage of exocrine cells (amylase positive) was observed during culturing, while the proportion of β-cells (insulin positive) rose significantly. Optimal maturation of pancreatic islets for transplantation occurred following 7 days of culture post-isolation, as they are highest in B cell concentration, with increased purity and viability from Day 3 of culture. While purity continues to increase post Day 7, islet function and B cell concentration does not significantly increase by Day 14, making islets most useful at Day 7.

### EXAMPLE 2

This study evaluates the changes in morphology and volume of alginate microcapsules cultured at predetermined temperatures to in order to determine optimal culture conditions for utilization in islet transplantation. Additionally, the effect of changes in incubation temperature on alginate microcapsules was evaluated by using dextrans of various molecular sizes to identify capsules with optimal diffusion parameters.

Experimental Design

Effect of Temperature on Microcapsule Morphology: Alginate microcapsules were synthesized from 2.5% (w/v) Ultra-Pure Low Viscosity Mannuronate (UP LVM; ≥85% mannuronic acid content) alginate or 2.5% (w/v) Ultra-Pure Low Viscosity Guluronate (UP LVG, NovaMatrix® PRONOVA™; ≥60% guluronic acid cont ent) alginate using an air-driven electrostatic generator (Nisco Engineering AG) at standard settings (Voltage: 9 kV, Agitator Speed: 80 rpm, Pressure: 3 psi, Needle gauge: 25 G, Needle height: 25 mm, Gelling solution: 120 mM Calcium Chloride). After encapsulation, the microcapsules were transferred to a 5 mM calcium chloride solution, analyzed for morphology and then stored for a 7-day period at the following temperatures: 3° C. and 37° C. After 7 days of in vitro culture at these temperatures, the microcapsules were analyzed for changes in their morphology and then transferred to different temperature conditions for an additional 7 days. At 12 hours, 24 hours, 3 days, 7 days, 10 days and 14 days, a minimum of 100 microcapsules were imaged under a phase contrast microscope for changes in morphology.

Effect of Temperature on Microcapsule Permeability: Blank and porcine islet-containing microcapsules were made with 1.5% (w/v), and 3.5% (w/v) ultra-pure low viscosity high mannuronate (UP LVM, NovaMatrix® PRONOVA™, Cat. 4200206, Lot. BP-0711-02) and ultra-pure low viscosity high guluronate (UP LVG, NovaMatrix® PRONOVA™, Cat. 4200006, Lot. BP-0710-04) using a microcapsule generator (Nisco Engineering AG, Zürich, Switzerland) at standard settings (9 kV; voltage, 80 rpm; stirrer speed, 3±10 psi; air pressure, 25±15 mm; needle height, 25 G; needle gauge, 120 mM CaCl₂; gelling solution). Microcapsules were either incubated at 24° C. or 37° C. in either 5 mM CaCl₂ or juvenile porcine islet (JPI) media (Optatio LLC, Irvine, Calif.) for 24-hours before plating the dextran diffusion assay.

A cocktail of three fluorescently-tagged dextrans was added to all wells in a 24-well plate along with 500 μL of either JPI or 5 mM CaCl₂ to match the storage media of the capsule tested. Dextran solutions were plated as follows: 10 μL (7 mg/mL) of cascade blue labeled 10 kDa dextran (Life Technologies, Cat. D-1976, Grand Island, N.Y.) 10 μL (7 mg/mL) of tetramethylrhodamine isothiocyanate (TRITC) labeled 500 kDa dextran (Sigma-Aldrich. Cat. 52194, St. Louis, Mo.), and 10 μL (7 mgl/mL) of either 70 kDa, 150 kDa, or 250 kDa fluorescein isothyocyanate (FITC) labeled dextran (Sigma-Aldrich, Cat. 90718, FD150S, FD250S, St. Louis, Mo.). Capsule samples were taken post-incubation and 500 μL suspensions were added to each well. Images were obtained (n=10) using a Leica TCS SP8 confocal microscope at 1, 30, and 60 minutes. Each color channel was run using sequential image recording to avoid fluorescent crosstalk. Microscope settings for each channel were set to maximize the number of gray values in the output signal of the photomultiplier (PMT) and the HyD detector. Leica HyD hybrid detectors were used for the red and green channel. A Leica PMT detector was used for the blue channel. The images were analyzed using a custom Graphical User Interface (GUI) made in house, which was written in MATLAB™ (v. 2014a, Mathworks, Natick, Mass. USA). The GUI primarily determined the percentage change in fluorescence intensity inside the capsules over time.

Results

Effect of Temperature on Microcapsule Morphology: Both UP LVM and UP LVG alginate microcapsules exhibited temperature-dependent, isotropic shrinkage over the study period. When compared to pre-incubation measurements (423.8±2.5 μm), microcapsules incubated for 14 days at physiological temperatures demonstrated significantly greater isotropic shrinkage than those incubated at lower temperatures (402.8±2.2 μm; 3° C. 398.1±1.5 μm; 23° C., 380.1±2.2 μm; 37° C., p<0.01). When microcapsules previously incubated at lower temperatures were transferred to physiological temperatures for an additional 7 days, as expected, they demonstrated significant isotropic shrinkage (410.8±2.0 μm→378.5±1.6 μm; 3° C.→37° C., 404.5±2.2 μm→375.3±1.9 μm; 23° C.→37° C., p<0.05). However, when the reverse was attempted, there was no significant change in microcapsule size (380.1±1.6 μm→378.5±1.6 μm; 37° C.→3° C., 380.1±1.6 μm→375.3±1.9 μm; 37° C.→23° C., (p=0.09).

Effect of Temperature on Microcapsule Permeability: Dextrans of all sizes were observed to begin diffusing immediately inside the capsules for each group at an increasing rate until reaching equilibrium, approximately one hour after plating (i.e., 1.5% UP LVG islet capsule in JPI at 37° C.; 10 kDa dextran; 1 min: 17±0%, 30 min: 62±1%, 60 min: 62±1%; 70 kDa dextran: 1 min; 15±0%, 30 min: 49±1%, 60 min: 49±1%: 150 kDa dextran; 1 min: 14±0%, 30 min: 42±1%, 60 min: 43±1%: 250 kDa dextran; 1 min: 13±0%, 30 min: 39±1%, 60 min: 41±1%; 500 kDa dextran; 1 min: 8±1%, 30 min: 34±1%, 60 min: 36 ±0%). As a general trend, 10 kDa dextrans showed the greates diffusion followed by 70 kDa dextrans, 150 kDa dextrans, 250 kDa dextrans, and 500 kDa dextrans, which demonstrated the least amount of diffusion in all groups, suggesting that alginate microcapsules are relatively impermeable to large protein molecules. Interestingly, the presence of islets in the capsules did not significantly affect diffusivity (i.e. 1.5% UP LVM capsule in JPI at 24° C.; 10 kDa dextran; 60 min; islet: 48±1% vs. blank: 48±0), indicating there were no tissue impurities in the capsule walls from the encapsulation method used. Dextrans showed decreased diffusion as alginate viscosity increased (i.e. 2.5% UP LVM blank capsule in 5 mM CaCl₂ at 37° C.: 150 kDa dextran; 60 min; 31±0% vs. 14±0% in 3.5% capsules), though diffusion was still significantly higher in capsules with high mannuronate content over high guluronate content (i.e. 2.5% UP LVM blank capsules in 5 mM CaCl₂ at 24° C.; 70 kDa dextran; 60 min; 35±0% vs. 66±0% in UP LVG capsules). Dextran diffusion decreased after exposure to JPI media (i.e. 3.5% UP LVG blank capsule in 5 mM CaCl₂ at 37° C.; 10 kDa dextran; 60 min; 65±0% vs. 38±0% in JPI), likely due to the media's sodium chloride content replacing calcium ions in the capsule walls. Both capsules in JPI media and 5 mM CaCl₂ showed decreased diffusion when stored at 37° C. compared to 24° C. (i.e. 1.5% UP LVG blank capsule in JPI; 10 kDa dextran; 60 min; 37° C.; 48±0% vs. 61±0% in 24° C. capsules), likely due to capsule shrinkage observed at higher temperatures causing tightening of the capsule walls.

Discussion and Interpretation

The alginate capsules exhibited time and temperature-dependent, isotropic shrinkage. By the 7-day time point, the diameter of capsules incubated at all temperatures was significantly different (p-value<0.01) than their original size, which correlated to decreases in volume. Microcapsules incubated at 3° C. displayed isotropic shrinkage when transferred to higher temperatures after 7 days, but those incubated at 37° C. and then transferred to lower temperatures did not show this phenomenon. The ability to optimize alginate microcapsule morphology is an important step in development of clinical-grade encapsulation strategies for the treatment of diabetes. Isotropic shrinkage noted at physiological temperatures is expected to affect microcapsule permeability, which has profound implications for encapsulated islet survival after transplantation. Assays that can evaluate microcapsule permselectivity were performed to determine the optimal culture conditions to achieve the ideal pore size to maximize transplant success in encapsulated islets. These results demonstrate that alginate composition (specifically, guluronic acid content) and temperature greatly influence permeability. These results further demonstrate that high mannuronate alginate microcapsules, higher viscosities, and higher temperatures confer significantly better protection from the humoral immune system. It is important to note that while capsules that were stored at 24° C. or in 5 mM CaCl₂ were studied here, encapsulated islets for transplantation should be stored in JPI media at 37° C. to preserve maximal tissue viability prior to transplantation. With this in mind, the encapsulated islets that showed the greatest promise for future transplants were both 3.5% UPLVG stored in JPI media at 37° C. and 2.5% UPLVM stored in JPI media at 37° C. While in most capsule types high mannuronate content showed better protection from 70 kDa to 250 kDa dextrans (representative of IgG antibodies), 3.5% UP LVG capsules stored in JPI at 37° C. demonstrated the same exclusion of these dextrans as their high mannuronate counterparts while imparting greater 10 kDa diffusion(representative of insulin), which is desirable for in vivo islet transplants.

### EXAMPLE 3

Although islet transplantation has been shown as a possible diabetes treatment, islets suffer acute hypoxic stress immediately following transplantation. It has been observed that up to 60% of newly-transplanted islets die during the first 48 hours post-transplantation as a direct result of hypoxic injury. This series of experiments demonstrates certain strategies and alginate microcapsule additives that may be used to increase encapsulated islet oxygenation and improve long-term islet viability post-transplantation.

Experimental Design

Islets were obtained from male Sprague Dawley Rats (300-350 g) and cultured in vitro overnight at 37° C., 5% CO₂. Supplemented CMRL media (Gibco) incubated at 0% O₂ overnight in a hypoxia chamber (XVivo, Biospherix) in order to allow media to deoxygenate. Dissolved oxygen levels were measured in the media using dissolved oxygen meter (Oakton D110).

Phosphate-complexed calcium peroxide (PcCPO) was synthesized by mixing calcium peroxide (CPO) with phosphate-buffered saline at 10 mg/mL and incubating at 37° C. overnight. CP or PcCP was dissolved in a 1.5% UPLVM solution and alginate microcapsules were produced as described above. All oxygen concentration assays were performed in a controlled hypoxic chamber at 37° C. 5% CO₂ 0% ambient O₂ to ensure that the data obtained was not confounded by changes in ambient O₂ during the experiment.

For all viability studies, islets were stained with the following probes to determine viability status based on positive staining: Calcein Blue: live; propidium iodide: dead; and YoPro-1; apoptotic. Islet function was evaluated by looking at glucose stimulated insulin response (GSIR). Islet stimulation index (insulin release at high glucose concentration/insulin release at low glucose concentrations) or SI was calculated after insulin release was quantified using an ELISA.

Results

In a first experiment, oxygen release from unencapsulated and encapsulated POGS, 10 mg/ml CPO in this case, was measured in vitro for a two hour period in the hypoxia chamber in culture media or in a 1:1 mixture of culture media and PBS. No islets were used in this experiment. As shown in FIG. 5, unencapsulated CPO added directly to the culture media (“POGS”) resulted in an immediate and sustained dissolved oxygen content of about 40%. When a 1:1 mixture of culture media and PBS was used, unencapsulated CPO (“PGS/PBS”) caused a rapid rise to 20% dissolved oxy gen which was sustained for at least an hour before gradually falling to 10%. Encapsulated CPO result ed in a lower but more stable level of dissolved oxygen over the study period with slightly higher levels generally observed when the media/PBS substrate (“POGS/PBS Encap”) was used relative to media alone (“POGS Encap”).

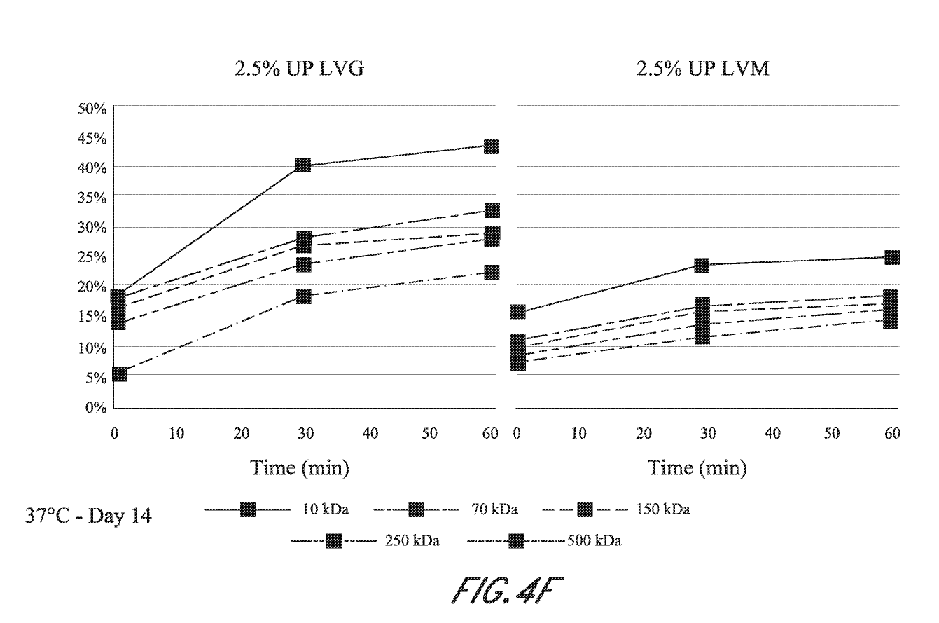

FIG. 6 shows the results of a second experiment which varied the encapsulated CPO concentration (5, 10, and 25 mg/ml) and also included an encapsulated PcCPO (5 mg/ml). Here again, the presence of PBS in the culture media prolonged and stabilized the oxygen-releasing effect of the CPO. Phosphate complexed-CPO resulted in a higher dissolved oxygen content than uncomplexed CPO.

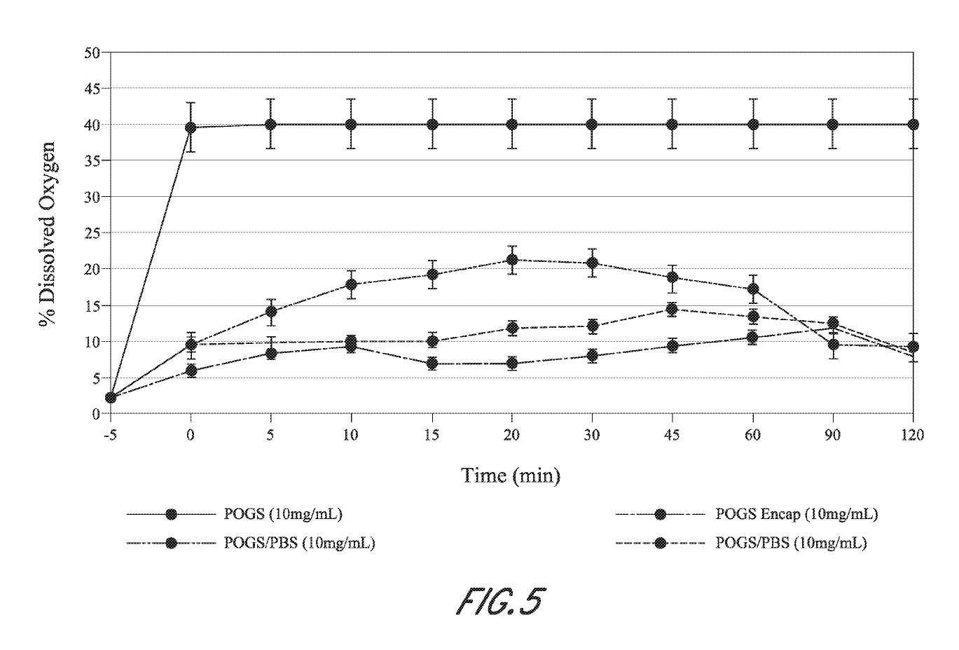

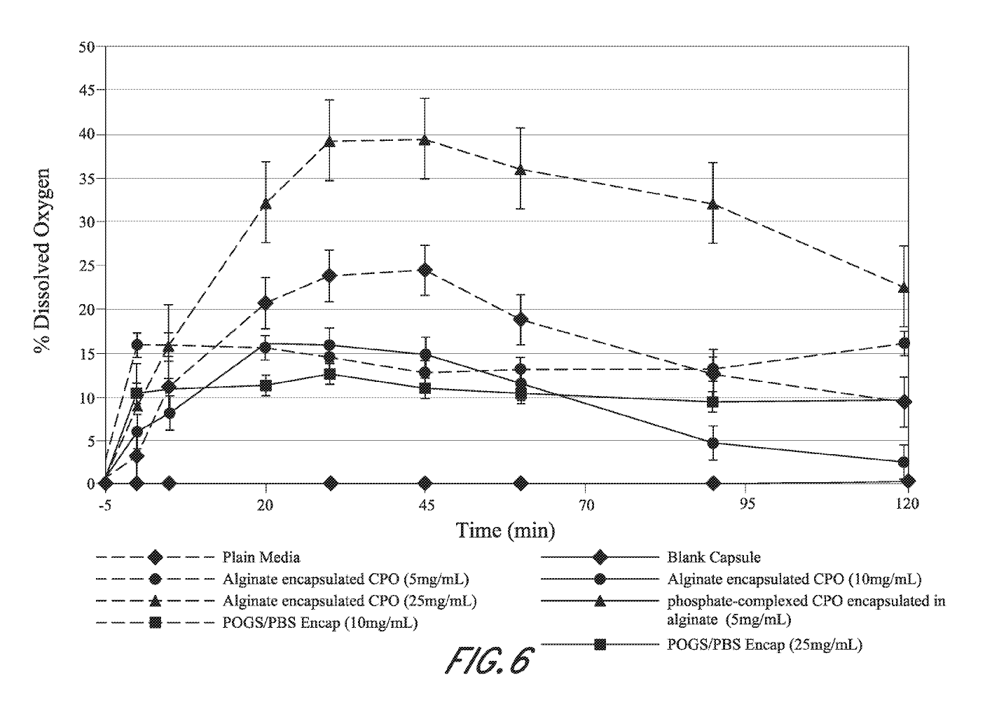

FIG. 7 shows the viability of isolated islets subjected to hypoxia in the presence of absence of CPO (“POGS”) in the culture media or a 1:1 mixture of culture media and PBS. About 60% and 70% of islets were alive and viable immediately after isolation and after the 24 hour culture at 20% O₂, respectively. However, the proportion of live/viable islets dropped to less than 30% after 24 hour culture at 0% O₂. Islets cultured for 24 hours at 0% O₂ were rescued by 5 mg/ml or 10 mg/ml CPO (“POGS”) in culture media or in the culture media/PBS mixture (p<0.01 relative to 0% O₂ without POGS).

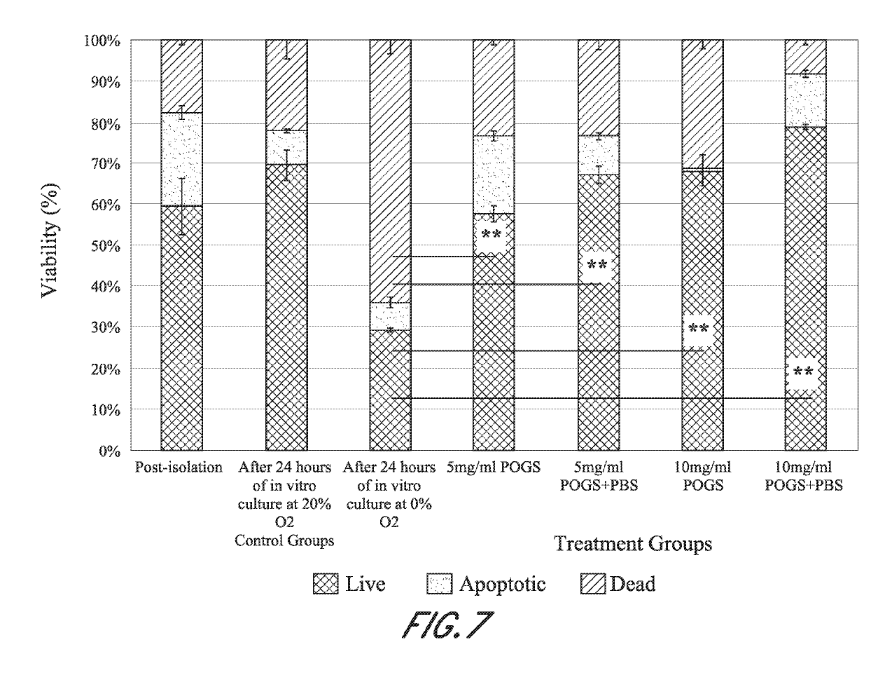

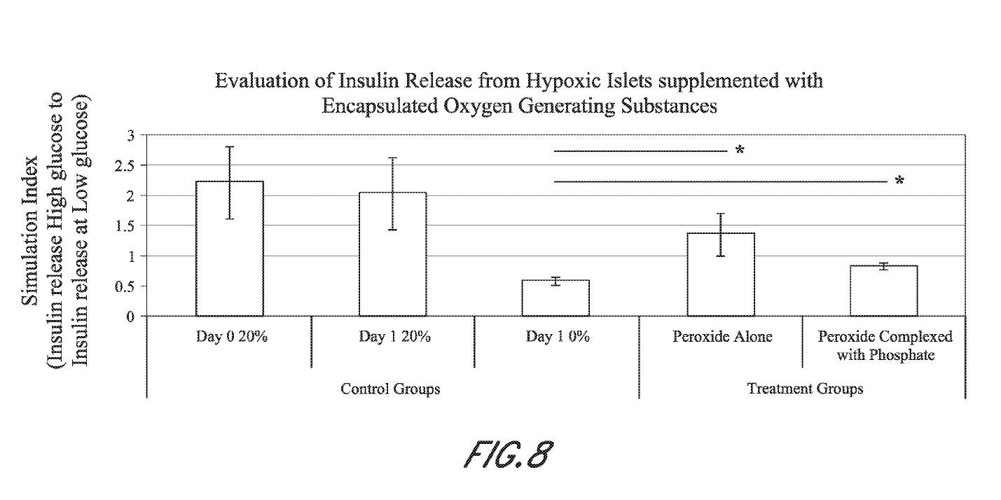

In order to be useful for diabetes treatment, the isolated islets must retain the ability to produce insulin, not just maintain viability. Thus, the insulin release characteristics of islets cultured at 0% O₂ in the presence of CPO was investigated by measuring the glucose-stimulated insulin release (GSIR). As shown in FIG. 8, islets cultured for 24 hrs at 0% O₂ showed a reduction of GSIR of about 75% relative to islets cultured at 20% O₂. The addition to the culture media of CPO or PcCPO significantly reversed this reduction (p<0.05).

Discussion and Interpretation

Although CPO and other POGS release oxygen to the surrounding media, the release profile is characterized by an initial burst, often producing hyperbaric conditions that can be fatal to transplanted islets. The results of these experiments demonstrate that encapsulation significantly blunts the initial oxygen burst from CPO. These results further demonstrate that CPO/POGS may be used as an oxygen source to maintain islet viability and insulin release characteristics under otherwise hypoxic conditions. POGS therefore may be usefully incorporated into microcapsules in order to maintain islet viability between the time of transplantation and vascularization/angiogenesis.

### EXAMPLE 4

Perfluorocarbons (“PFCs”) are organic compounds with fluorine-substituted hydrocarbon chains that have an intrinsic affinity for oxygen and other gases, often many times higher than hemoglobin. PFCs are chemically inactive compounds that are generally nontoxic to humans in modest doses. This study investigated whether the addition of perfluorocarbons to an islet culture can provide sufficient oxygen directly to islets to maintain viability and critical function. The goal of the study is to understand whether the incorporation of PFCs into alginate microcapsules can preserve islet viability and function by eliminating low oxygen diffusion rate that is characteristic of the microcapsules immediately after transplantation.

Experimental Design

Islets from Sprague Dawley rats were isolated, cultured, and assessed as described above. Three concentrations of perfluorodecalin (“PFD”) were added to the culture media: 0.1 ml PFC/ml media (“low PFC”), 0.375 ml PFC/ml media (“medium PFC”), and 0.75 ml PFC/ml media (“high PFC”).

Results

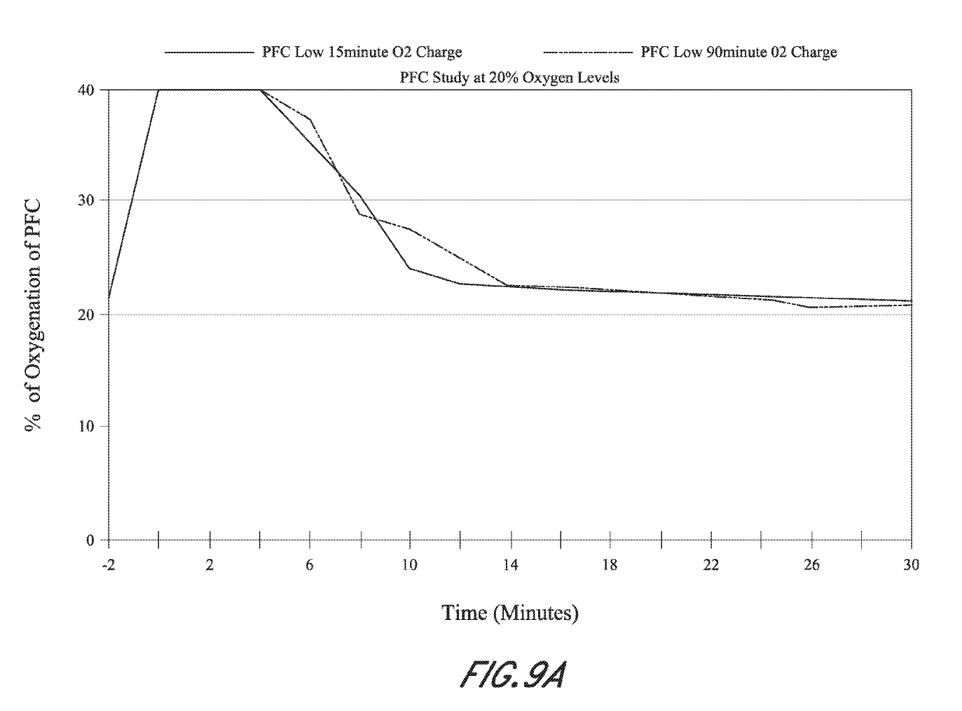

A first experiment was performed under 20% and 0% oxygen culture conditions in the absence of islets to characterize the oxygen release rates of various PFC concentrations and under different PFC-oxygen charging conditions. FIG. 9A shows the oxygen content of PFD when charged for either 15 to 90 minutes in 40% O₂ before being added to culture media at 20% O₂. The oxygen content of the PFD fell from 40% to the ambient 20% within 10 minutes and the oxygen charging time did not have a significant effect on the time-dependent release profile. FIG. 9B shows the time-dependent oxygen release from the PFC under hypoxic conditions. The PFCs were charged for 15 minutes in 40% O₂ prior to plating. These results demonstrate that PFC maintain a prolonged O₂ release profile under hypoxic conditions.

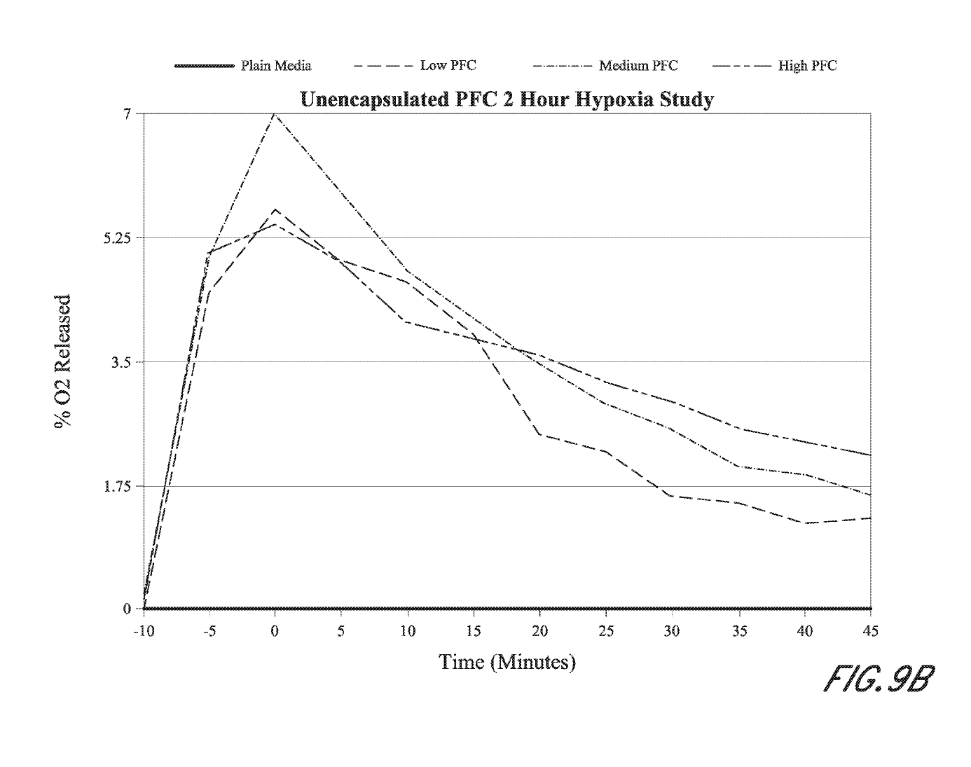

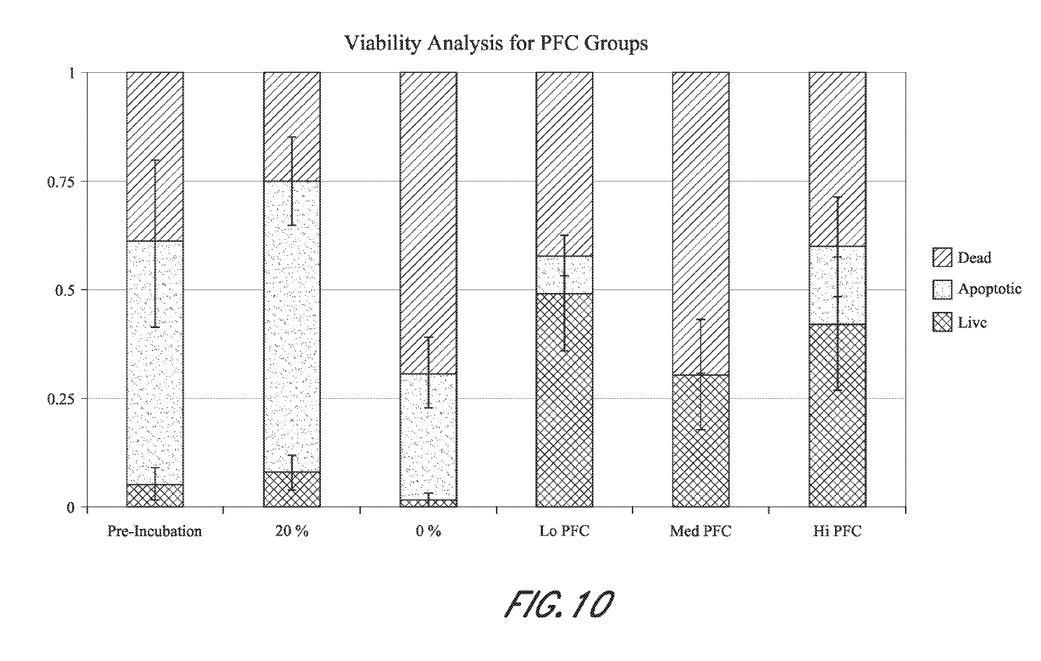

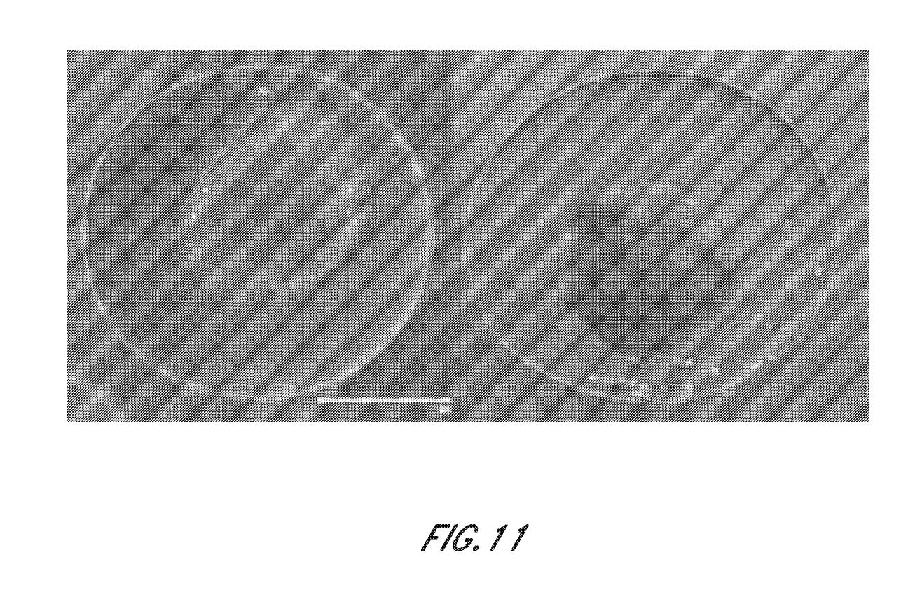

In a second experiment, islets were plated under 0% or 20% oxygen conditions, as outlined above. Hypoxic islets were supplemented with low, medium, or high PFC levels, charged for 15 minutes in 40% O₂ prior to plating. Islet viability was assessed as described above. FIG. 10 and the table 1 demonstrate that, under hypoxic conditions, all PFC concentrations significantly improved islet viability at 24 hours.

TABLE 1
Effect of PFCs on Islet Viability in Hypoxic Culture

| | | Live (%) | Apoptotic (%) | Dead (%) |
|---|---|---|---|---|
| 20% O₂ | Pre-incubation | 5.36 | 55.2 | 39.4 |
| | 24 hrs. | 7.96 | 67.0 | 24.9 |
| 0% O₂ | Control | 1.69 | 29.3 | 69.0 |
| | Low PFC | 49.2 | 8.73 | 42.1 |
| | Medium PFC | 30.5 | 0.355 | 69.1 |
| | High PFC | 42.3 | 17.7 | 39.9 |

Discussion and Interpretation

These data demonstrate that PFCs, particularly PFD, may be used to capture and release oxygen in order to sustain islet viability under hypoxic conditions. It is known that many PFCs have a high affinity for oxygen. Therefore, PFCs and POGS may be combined in an alginate microcapsule to further improve islet viability after transplantation. The PFC may be used to absorb the initial oxygen burst from the POG, thereby protecting the encapsulated cells from oxidative stress and then to provide a sustained release of the absorbed oxygen over time.

### EXAMPLE 5

The following experiment was performed to investigate whether an immunorepellant retained and expressed a measurable biological activity when incorporated into a microcapsule.

Porcine islet cells were encapsulated to 2.5% UPLVM microcapsules using the methods described above. One set of microcapsules incorporated CXCL12/SLF-1 to investigate the locally immunosuppressive effects of this immunorepellant. Microcapsules were then cultured in the presence of macrophages.

FIG. 6 are representative photomicrographs of a microcapsule containing CXCL12/SLF-1 (left) and a control microcapsule lacing CXCL12/SLF-1 (right). Macrophage attachment was observed only for the microcapsules lacking CXCL12/SLF-1. These data demonstrate that immunorepellants may be used for local immunosuppression and to prolong the viability of transplanted microencapsulated islets.

It will be appreciated by persons having ordinary skill in the art that many variations, additions, modifications, and other applications may be made to what has been particularly shown and described herein by way of embodiments, without departing from the spirit or scope of the invention. Therefore it is intended that scope of the invention, as defined by the claims below, includes all foreseeable variations, additions, modifications or applications.

What is claimed is:

1. An alginate microcapsule having a diameter of 250-600 μm and comprising at least one of (a) immunorepellant, (b) a particulate oxygen generating substance, and (c) a perfluorocarbon.

2. The microcapsule of claim 1, wherein the alginate is a low viscosity alginate.

3. The microcapsule of claim 1 or 2, wherein the alginate comprises at least 50% guluronic acid.

4. The microcapsule of claim 1 or 2, wherein the alginate comprises at least 70% mannuronic acid.

5. The method of any one of claims 1-4, wherein the microcapsule further comprises an angiogenic molecule.

6. The microcapsule of claims 5, wherein the microcapsule comprises an angiogenic molecule selected from the group consisting of fibrin, FGF-1, FGF-2, VEGF, PDGF, and MMP.

7. The microcapsule of any one of claims 1-6, wherein the microcapsule comprises immunorepellant selected from the group consisting of CXCL12, SDF-1, IL-8, and TGF-β.

8. The microcapsule of any one of claims 1-7, wherein the microcapsule comprises a particulate oxygen generating substance selected from the group consisting of inorganic peroxides and inorganic percarbonates.

9. The microcapsule of claim 8, wherein the particulate oxygen generating substance is selected from the group consisting of calcium peroxide and sodium percarbonate.

10. The microcapsule of any one of claims 1-9, wherein the perfluorocarbon is perfluorodecalin.

11. The microcapsule of any one of claims 1-10, wherein the microcapsule further comprises a viable population of therapeutic cells.

12. The microcapsule of claim 11, wherein the population of therapeutic cells comprises cells selected from the group consisting of islet cells, stem cells hepatocytes, chondrocytes, and lipocytes.

13. The microcapsule of claim 11 or 12, wherein the population of therapeutic cells secretes a biologically active molecule.

14. The microcapsule of any one of claims 11-13, wherein the population of therapeutic cells comprises islet cells.

15. The microcapsule of claim 14, wherein the islet cells are human islet cells or porcine islet cells.

16. The microcapsule of any one of claims 13-15, wherein the biologically active molecule is insulin.

17. A method for treating diabetes in a subject, the method comprising:
(a) diagnosing the subject as having diabetes; and
implanting into the subject a composition comprising the microcapsules of any one of claims 11-16, wherein the population of therapeutic cells secrete insulin.

18. The method of claim 17, wherein the composition is implanted subcutaneously.

19. The method of claim 17, wherein the composition is implanted within the intraperitoneal cavity.

20. The method of any one of claims 17-19, wherein the subject is a human, cat, or dog.
